(19) United States
(12) Patent Application Publication
(10) Pub. No.: US 2021/0236269 A1
(43) Pub. Date: Aug. 5, 2021

(54) OPHTHALMOLOGICAL IMPLANT, METHOD FOR PRODUCING AN OPHTHALMOLOGICAL IMPLANT, AND USE OF A LIGAND FOR PRODUCING AN OPHTHALMOLOGICAL IMPLANT

(71) Applicant: Carl Zeiss Meditec AG, Jena (DE)

(72) Inventor: André Wolfstein, Berlin (DE)

(21) Appl. No.: 17/239,305

(22) Filed: Apr. 23, 2021

Related U.S. Application Data
(63) Continuation of application No. PCT/EP2019/075325, filed on Sep. 20, 2019.

(30) Foreign Application Priority Data
Oct. 26, 2018 (DE) 10 2018 126 842.4

(51) Int. Cl. A61F 2/16; C07K 16/22; C07K 16/24; A61K 47/69; A61L 27/54; A61L 27/20; A61L 27/18

(52) U.S. Cl. CPC A61F 2/1624; C07K 16/22; C07K 16/245; A61K 47/6957; A61L 2430/16; A61L 27/20; A61L 27/18; A61F 2240/001; A61F 2002/1681; A61L 27/54

(57) ABSTRACT

The disclosure relates to an ophthalmological instrument with a main body and at least one ligand (L) immobilized on the main body. In the implanted state of the ophthalmological implant, the ligand (L) binds and/or deactivates at least one fibrinogen and/or cytokine. The disclosure further relates to a method for producing an ophthalmological implant, and to a use of a ligand (L), via which at least one fibrinogen and/or cytokine is to be bound and/or deactivated in the implanted state of the ophthalmological implant, for producing an ophthalmological implant.

### CROSS REFERENCE TO RELATED APPLICATIONS

This application is a continuation application of international patent application PCT/EP2019/075325, filed Sep. 20, 2019, designating the United States and claiming priority from German application 10 2018 126 842.4, filed Oct. 26, 2018, and the entire content of both applications is incorporated herein by reference.

### TECHNICAL FIELD

The disclosure relates to an ophthalmological implant, to a method for producing an ophthalmological implant, and to use of a ligand for producing an ophthalmological implant.

### BACKGROUND

Fibrosis refers to a pathological increase in connective tissue in human and animal tissues, the main component of which is collagen fibers. The tissue of the organ affected is hardened as a result. What arise are scarred changes which, at an advanced stage, lead to restriction of the particular function of the organ. In the case of use of ophthalmological implants, for example intraocular lenses, interactions between the implant and adjacent biological tissue mean that various complications can occur, of which such fibroses in particular are problematic. For example, so-called posterior capsule opacification (PCO, cataracta secundaria) occurs in some cases after cataract operations. PCO is a postoperative clouding of the lens capsule after surgical extraction of a natural lens. The remaining lens epithelial cells (E cells) in the equatorial region of the capsular bag are mitotically active and can transform into fibroblasts. These then trigger a kind of wound healing, involving formation of collagen-containing connective tissue.

Since some fibroblast subtypes not only migrate onto the inner side of the capsular bag, but can also contract, wrinkles form in the capsular bag. The clouding of the capsule is therefore the result of a wound healing process and associated scarring. Since the lens clouding caused thereby has causes other than the original cataract disease, this is referred to as an “aftercataract” or a “secondary cataract”. For those affected, a clinically significant aftercataract can lead to a reduction in visual acuity, in color perception, and in contrast vision, and to increased glare.

Secondary cataract is a common complication after extracapsular cataract extraction (ECCE) and the subsequent implantation of an intraocular lens (IOL) in the capsular bag. Without the implantation of an IOL in the empty capsular bag, the risk of an aftercataract is even more considerably increased, since unhindered cell migration to the posterior surface of the capsular bag is possible in this case.

The incidence of a secondary cataract increases over time after a surgical procedure. A meta-analysis of the cases of cataracta secundaria for all existing types of IOLs showed an approximate average increase of 12% one year after surgery and an approximate average increase of 30% five years after surgery. The age of the affected patient also appears to be a crucial factor here, and so it has to be expected that almost all treated children and adolescents might suffer from a postoperative secondary cataract after a certain period of time.

It has so far not been possible to quantitatively remove all epithelial cells during eye operations without using toxic drugs such as, for example, 5-fluorouracil, thapsigargin, or paclitaxel, which can damage the endothelial cell layer of the cornea or other eye tissue. The vision problems caused by fibrosis and PCO are partially treated by so-called focal Nd:YAG capsulotomy. Nevertheless, fibrotic striae in particular might continue to impair the function of accommodative IOLs.

### SUMMARY

Disclosed is an ophthalmological implant that reduces the risk of PCO and fibrosis. Disclosed are also methods of producing such an ophthalmological implant.

Provided is an ophthalmological implant having a main body and at least one ligand immobilized on the main body through which at least one fibrinogen and/or cytokine is bound and/or deactivated when the ophthalmological implant has been implanted. In other words, the described ophthalmological implant or its main body has at least one ligand or ligand type immobilized with respect to the main body, that is to say spatially fixed on the main body. In general, “a/an” in the context of this disclosure is to be read as an indefinite article, that is, always as “at least one” if there is no express indication of the contrary. For example, a mixture of two or more different ligand types can be provided. Conversely, “a/an” can also be understood to mean “just one”. For example, only a single ligand type can be provided. In the context of the present disclosure, a ligand is understood to mean a molecule which can specifically bind to a target structure and/or can specifically react or interact with the target structure in order to deactivate the biological functionality of the target structure by, for example, binding, inhibition, deactivation and/or reaction. To this end, the ligand can, for example, alter the target structure enzymatically, that is, for example act as a hydrolase, isomerase, ligase, lyase, transferase and/or oxidoreductase, or crosslink the target structure or alter the target structure in some other way in order to prevent its biological functionality. Alternatively or additionally, in another embodiment, the ligand functions as an agonist or antagonist or comprise a paratope, through which an epitope of a target structure is bound. The binding of the ligand to the target structure is in some embodiments, in principle, reversible or irreversible and in some embodiments is brought about by ionic bonds, hydrogen bonds, van der Waals forces, hydrophobic effects or any combinations thereof. Furthermore, in some embodiments the ligand binds the target structure without modification or chemically modifies the target structure. The affinity of a ligand for the target structure is in some embodiments determined with the aid of conventional ligand-binding tests. In the present case, the target structure of the at least one ligand is at least one fibrinogen and/or one cytokine, with which the ophthalmological implant comes or can come into contact when it has been implanted. In the present case, fibrinogens refer to substances and cells which cause fibrosis. In the present case, cytokines refer to substances that initiate and/or regulate the growth and/or the differentiation of cells in the human and animal eye. In this way, the ophthalmological implant described herein, after its implantation in a human or animal eye, binds fibrinogens and/or cytokines and/or deactivates their biological function, with the result that the transformation and proliferation of remaining lens epithelial cells is prevented, thus preventing the occurrence of fibrosis, PCO, and similar diseases. The presence of the at least one ligand or ligand type on the main body in an immobilized state reliably prevents dilution or wash-out of the ligand, for example by aqueous humor, and as a result, the antifibrotic and/or anti-cytokine action of the implant described herein can be maintained over a very long period of time, even in contrast to an active-substance delivery system (drug delivery system) for example.

In an embodiment, the ophthalmological implant described herein is configured as an intraocular lens (IOL), in particular an accommodating intraocular lens, or as a ring, in particular a capsular tension ring. As a result, the advantages realizable therewith are realized in the context of different eye operations and implant types. This applies especially to an implant configured as an accommodating IOL, since, in the case of such an IOL, fibrosis that occurs might impair or completely prevent the accommodation ability of the IOL. Furthermore, the ophthalmological implant described herein, in an embodiment, is configured as a tube, in particular as a dialysis tube.

In a further embodiment, the ligand has been embedded in a matrix. This provides a simple way of immobilization via the mechanism of steric hindrance. For this purpose, the matrix has a three-dimensional lattice with cavities in which molecules of the ligand type have been arranged, the ligand molecules being too large to escape through the gaps in the lattice. The ligand thus permanently remains inside the matrix, in contrast to a drug delivery system, in the case of which active substances are to be deliberately delivered from a matrix into the surroundings. It is understood that the gaps in the lattice of the matrix should, conversely, be large enough so that the target structures of the ligand can pass through and interact with the ligand. In order to adjust the size or the volume of the ligand so that it reliably remains inside the matrix, the ligand in an embodiment is coupled to an appropriately large molecule. If the ligand is, for example, an antibody that is too small for the matrix in question, the ligand can, for example, be designed as a fusion protein composed of the antibody and a further protein and be immobilized in the matrix. Furthermore, the ligand has in an embodiment covalently bonded to it at least one polymer, in particular to at least one biopolymer. As a result, immobilization in the assigned matrix is likewise achieved. Alternatively in another embodiment, the ligand covalently bonded to the polymer is used without being embedded in a matrix, if the polymer itself—and thus also the covalently bonded ligand—has been immobilized on the main body. In an advantageous embodiment, the main body comprises, at least regionally, the polymer covalently coupled with at least one ligand. In other words, the main body completely, predominantly or partially is made of the polymer to which the ligand has been covalently coupled. In the context of the present disclosure, a polymer is understood to mean macromolecules that have been synthesized from one or more structural units (repeat units). The polymer is generally made of identical or nonidentical macromolecules and is a synthetic or semisynthetic polymer or a biopolymer. In the context of the present disclosure, a biopolymer is understood to mean polymers that are synthesized in the cell of an organism and also polymers comprising biogenic raw materials (renewable raw materials) and/or are biodegradable (biogenic and biodegradable polymers). The term biopolymer thus includes biobased biopolymers that are biodegradable or non-biodegradable and also petroleum-based polymers that are biodegradable.

In a further embodiment, the at least one ligand has been covalently coupled to the at least one polymer via a spacer, that is to say via a structural unit acting as a spacing element. Firstly, in such an embodiment, the ligand is thereby bound in a particularly simple manner to the particular reactive groups present in the polymer and, secondly, the binding capacity of the ligand is thereby improved, since, for example, any steric hindrances that might hinder interaction with the target structure is avoided by choosing a suitable spacer.

In a further embodiment, the at least one polymer is a polysaccharide selected from cellulose, a cellulose ether with methyl and/or ethyl and/or propyl groups, in particular hydroxypropylmethylcellulose, hydroxyethylmethylcellulose, and/or methylcellulose, a glycosaminoglycan, in particular hyaluronic acid, chondroitin sulfate, dermatan sulfate, heparin, heparan sulfate, keratan sulfate, alginic acid, polymannuronic acid, polyguluronic acid, polyglucuronic acid, amylose, amylopectin, callose, chitosan, polygalactomannan, dextran, xanthan gum, and/or a mixture, and/or a physiologically acceptable salt, in particular alkali metal salt, thereof. As a result, the properties, for example the viscoelasticity, can be optimally adapted to the particular intended use and purpose of the implant. Polysaccharides, which are also referred to as glycans, are in general a subclass of carbohydrates and are polysugars composed of monosaccharide units (for example, glucose, fructose, galactose, et cetera) that form a chain. Every monosaccharide, which is also referred to as simple sugar, is made of a chain of carbon atoms. Depending on the number of carbon atoms, reference is made to trioses (3), tetroses (4), pentoses (5), hexoses (6), heptoses (7), et cetera. Depending on the nature of their saccharide units, the polysaccharides are divisable into homoglycans, which have only one type of simple sugar, and heteroglycans, which have two or more different types of simple sugars. Furthermore, polysaccharides are either unsubstituted or substituted and optionally bear one or more side groups such as, for example, hydroxyl, carboxy, amino, or sulfate groups.

In a further embodiment, the ophthalmological implant comprises a main body having at least one haptic part and at least one optical part, wherein the at least one ligand has optionally been arranged at least on the haptic part. The ligand has in some embodiments been completely arranged in or on the haptic part. This reliably prevents the functionality of the optical part from being impaired by the ligand and any other compounds. Furthermore, there is the possibility of configuring the haptic part as a physical barrier against cell migration, by exerting a predetermined pressure on the surrounding tissue, for example on the capsular bag, when the ophthalmological implant has been implanted. A curved haptic part thus allows significantly increased contact with the surrounding tissue and thus additionally prevents infiltration by cells. Although the main body can in principle also be one-piece, depending on the embodiment of the ophthalmological implant, the embodiment with a haptic part and an optical part offers the further advantage that the main body can have been produced from different materials.

Alternatively or additionally, in some embodiments, the main body comprises an accommodation region in which the immobilized ligand has been arranged. For example, in such embodiments, the main body has a pocket or the like, inside which the ligand is present in an immobilized state. As a result, the ligand is optimally arranged depending on the particular intended purpose of the implant. Alternatively or additionally, the at least one ligand has been covalently bonded to the main body. This direct or indirect connection ensures particularly reliable immobilization. For example, a crosslinker such as, for example, 1,4-butanediol diglycidyl ether is used for covalent bonding. Depending on the particular material of the main body, it is, however, naturally also possible to use other suitable crosslinkers or mixtures thereof.

In a further embodiment, at least part of a surface of the main body has been coated with a layer system that comprises the immobilized ligand. In other words, the main body, which for its part is completely or partially made of one or more polymers, is in some embodiments at least partially or completely coated with a layer system, wherein the layer system for its part is made of one layer or of two or more layers and wherein at least one of the layers comprises the at least one ligand or consists of the at least one ligand. The layer system is thus, in principle, in such embodiments, covering the entire surface of the main body or merely one or more selected surface regions of the main body. Similarly, the layer system is in some embodiments made of two layers that comprise or consist of the at least one ligand, wherein the at least two layers can have the same composition or different compositions. As a result, effects that are staggered in terms of time and/or location can be realized.

In a further embodiment, the layer system comprises at least two layers, wherein at least one of the at least two layers comprises the at least one ligand and at least one of the other layers comprises a polymer selected from polyethylenimines, polyamines, and polyallylamines. As a result, surface-charged layers can be produced.

Further advantages arise from the fact that the at least one ligand binds and/or deactivates at least one fibrinogen and/or cytokine, in particular TGF-β, TNF-α, and/or interleukin-1, occurring in the aqueous humor. As a result, the disadvantageous effects of fibrinogens and/or cytokines occurring in aqueous humor are avoided in a therapeutically effective manner. In the context of the present disclosure, the expression “TGF-β” implicitly also encompasses the isoforms TGF-131, TGF-β2, TGF-β3, and TGF-β4. The levels of fibrinogens and/or cytokines in the aqueous humor increase after a surgical trauma due to the implantation of the implant and generally only fall back to their starting values after months. Owing to the permanent removal of fibrinogens and/or cytokines via the ligand, the disadvantageous effects of these substances are reliably prevented over the required, comparatively long period of several months, since, for example, stable transdifferentiation of the epithelial cells into fibroblast-like cells which cause PCO and fibrosis is not possible. This is particularly important for accommodative IOLs, the functionality of which is particularly strongly impaired by fibrosis.

In a further embodiment, the at least one ligand is selected from antibodies, in particular anti-TGF-β antibodies, anti-TGF-α antibodies, and/or anti-interleukin-1 antibodies, Fab fragments, single-chain variable fragments (scFv), and multivalent antibody fragments (scFv multimers). As a result, in such an embodiment, a selection is performed that is optimally adapted to the fibrinogen and/or cytokine to be removed. Antibody fragments offer the advantage of a high binding affinity/avidity and specificity for a broad spectrum of target structures and haptens. Single-chain fragments can moreover be crosslinked or expressed as diabodies (60 kDa), triabodies (90 kDa), tetrabodies (120 kDa), et cetera, with different linker lengths between V domains being possible. Moreover, a particular advantage is that molecules of 60-120 kDa increase the penetration of cells and have faster clearance rates than corresponding Igs (150 kDa). Furthermore, in an embodiment, the ligand is a diabody, triabody, tetrabody, pentabody, hexabody, heptabody, or octabody. In other words, the ligand is monospecific, bispecific, trispecific, tetraspecific, pentaspecific, hexaspecific, heptaspecific, octaspecific, nonaspecific, or multispecific. This allows the crosslinking of two, three, four, five, six, seven, or eight target structures or target proteins, it being possible for scFv multimers to be adapted particularly precisely and individually to the spatial arrangement of the target epitopes of certain fibrinogens and/or cytokines, which spatial arrangement may be patient-specific. The increased binding valency of scFv multimers leads to a particularly high avidity.

Disclosed are also methods for producing an ophthalmological implant, in which a main body having at least one ligand immobilized on the main body, through which at least one fibrinogen and/or cytokine is able to be bound and/or deactivated when the ophthalmological implant has been implanted, is produced. As a result, an ophthalmological implant that reduces the risk of PCO and fibrosis is created. Further features arising therefrom and the advantages thereof can be gathered from the descriptions provided herein.

In an embodiment, it has proved advantageous that the at least one ligand is covalently coupled to a polysaccharide. Covalent coupling is achieved in some embodiments by: providing the ligand having at least one amino group, providing the polysaccharide having at least one carboxylic acid group, at least partially activating the carboxylic acid group of the polysaccharide, and coupling the activated carboxylic acid group with the at least one amino group of the ligand. As a result, antibodies, antibody fragments, and the like in particular, which regularly have multiple amino groups, are covalently bonded to the polysaccharide containing carboxylic acid groups. The partial or complete activation of the carboxylic acid group(s) present in the polysaccharide are, for example, achieved with the aid of N-hydroxysulfosuccinimide (NHS/sulfo-NHS), meaning that, in an aqueous solution, an optionally carbodiimide-mediated (for example, 1-ethyl-3-(3-dimethylaminopropyl)carbodiimide hydrochloride/EDC, and/or N′,N′-dicyclohexylcarbodiimide/DCC) covalent coupling at, in an embodiment, physiological pH values.

Further advantages arise if a layer system having at least two layers is generated on a surface region of the main body, wherein at least one of the layers comprises the immobilized ligand and at least one of the other layers comprises a polymer selected from polyethylenimines, polyamines, and polyallylamines. In other words, a layer system composed of two or more layers is generated on one or more regions of the surface of the main body or on the entire main body, wherein at least one layer comprises or consists of the ligand, which can for example have been covalently coupled with a polysaccharide or another polymer, and at least one further layer comprises another polymer selected from polyethylenimines, polyamines, and polyallylamines, or consists of one of the polymers, or a mixture thereof. The layer system in some embodiments has a sandwich structure having multiple alternating layers of the ligand, optionally coupled with a polysaccharide, and the other polymer. The cover layer of the layer system in some embodiments is made of the ligand or a polymer, in particular a polysaccharide, covalently coupled with the ligand.

In a further embodiment, the main body is provided with an active-substance delivery system designed to deliver alkali metal ions selected from Li⁺, K⁺, Rb⁺, and/or Cs⁺, when the ophthalmological implant has been implanted. In other words, the ophthalmological implant described herein, or its main body, is provided with not only the immobilized ligand, but also with an active-substance delivery system for alkali metal ions, with the exception of Na⁺ ions (and radioactive Fr ions), which active-substance delivery system is herein, in contrast to the immobilized ligand, referred to as a drug delivery system. Alkali metal ions selected from Li⁺, K⁺, Rb⁺, and Cs⁺, additionally considerably slow down or even completely prevent the development of PCO and fibrosis, efficacy generally being the highest for Li⁺. According to current knowledge, sodium ions have no efficacy whatsoever and cannot prevent the development of PCO and fibrosis. Therefore, in the context of the present disclosure, the term “alkali metal ions” is generally understood to mean Li⁺, K⁺, Rb⁺, and Cs⁺, but not Na⁺ and Fr ions, unless Na⁺ and Fr ions are explicitly mentioned. The mentioned alkali metal ions Li⁺, K⁺, Rb⁺, and Cs⁺, are in various embodiments be delivered individually or in any combination. For example, in an embodiment the active-substance delivery system can deliver only Li⁺ ions or only Rb⁺ ions. In another embodiment, the active-substance delivery system is designed to deliver Li⁺ ions and K⁺ ions, and so on. Alkali metal ions of the group mentioned have the advantage that they—individually and in any combination—have no toxicity or only a very low toxicity, in particular for eye tissue, in the concentrations required for prevention of PCO and fibrosis. Without wishing to be bound by theory, it is assumed that topically administered alkali metal ions from the group mentioned are capable of preventing the conversion of equatorial epithelial cells which were not removed during an eye operation into fibroblasts, fibroblasts being, as already mentioned, the main cause of PCO and fibrosis. The effect is presumably based on the ability of alkali metal ions of the group mentioned to stabilize the quiescent, polarized phenotype of lens epithelial cells. The epithelial cells in question therefore maintain the apical-basolateral polarity and cobblestone-like arrangement that are characteristic of lens epithelial cells. When applied locally, the alkali metal ions of the group mentioned also block the potent epithelial mesenchymal transition (EMT)-promoting effect of transforming growth factor beta (TGF-β) on lens epithelial cells. Experiments have shown that cells in TGF-β-treated explants progressively dissociated away from one another and assumed various elongated spindle shapes. Furthermore, the cells expressed α-SMA (apha-actin-2, alpha smooth muscle actin) to a considerable extent. These features are characteristic of the development of PCO. As a result of treatment with alkali metal ions of the group mentioned, it was possible to block the formation of α-SMA effectively and to stabilize the cells in the polarized, adherent, cobblestone-like monolayer. The counterions (anion) which can be used in relation to the alkali metal ion(s) (cation) used are in principle all suitable or pharmaceutically acceptable anions formed from atoms and/or molecules. Particular preference is given to chloride ions (CI). In an embodiment, the active-substance delivery system is general exclusively made of a compound containing alkali metal ions of the group mentioned. Alternatively, in other embodiments, further components are provided, for example components for adjusting active-substance delivery or components in the form of binding or release agents. Furthermore, in another embodiment, the active-substance delivery system is a layer or part of a layer of a layer system.

Also described herein is the use of a ligand, through which at least one fibrinogen and/or cytokine is bound and/or deactivated when the ophthalmological implant is implanted, for production of an ophthalmological implant. As a result, an ophthalmological implant that reduces the risk of PCO and fibrosis is created. Further features arising therefrom and the advantages thereof can be gathered from the descriptions provided above.

The features and combinations of features mentioned in the description above and the features and combinations of features mentioned in the description of the figures below and/or shown in the figures alone are usable not only in the respectively specified combination, but also in other combinations, without departing from the scope of this disclosure. Hence, embodiments that are not explicitly shown and explained in the figures, but that emerge and are producible by way of separated combinations of features from the explained embodiments, are also considered to be encompassed and disclosed herein. Embodiments and combinations of features that therefore do not have all the features of an originally worded independent claim are also considered to be disclosed herein. Furthermore, embodiments and combinations of features that go beyond or deviate from the combinations of features set out in the dependency references of the claims are considered to be disclosed herein, in particular by virtue of the embodiments set out above.

### BRIEF DESCRIPTION OF THE DRAWINGS

The invention will now be described with reference to the drawings wherein:

FIG. 1 shows a schematic side view of part of an ophthalmological implant known from the prior art;

FIG. 2 shows an enlarged depiction of the detailed area II shown in FIG. 1;

FIG. 3 shows an enlarged depiction of the detailed area III shown in FIG. 1;

FIG. 4 shows a depiction of the principle of a reaction for covalently coupling a ligand to a polysaccharide;

FIG. 5 shows a chemical formula of the polysaccharide coupled with the ligand;

FIG. 6 shows a schematic lateral sectional view of one embodiment of an ophthalmological implant according to the invention; and,

FIG. 7 shows a schematic view of part of the ophthalmological implant according to the invention, which has immobilized thereon the polysaccharide coupled with the ligand.

### DESCRIPTION OF THE PREFERRED EMBODIMENTS

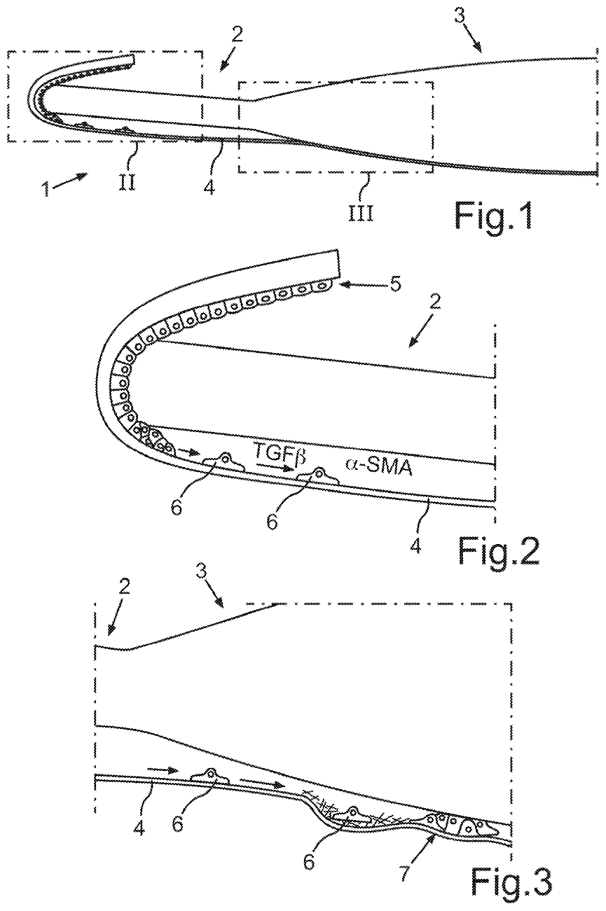

FIG. 1 shows a schematic side view of part of an ophthalmological implant 1 known from the prior art that, in the present case, is configured as an intraocular lens. The implant 1 comprises, in a manner known per se, a haptic part 2 and an optical part 3. For better understanding, FIG. 2 shows an enlarged depiction of the detailed area II shown in FIG. 1, whereas FIG. 3 shows an enlarged depiction of the detailed area III shown in FIG. 1. It can be seen that the implant 1, in particular in the region of the haptic part 2, does not lie continuously flush against the posterior capsular bag 4, since the capsular bag 4 forms folds and cavities. As schematically depicted in FIG. 2, the result of this is that so-called E cells 5, which are located in the equatorial region of the capsular bag 4, are mitotically active and normally form a cobblestone-like monolayer, progressively dissociate away from one other under the influence of transforming growth factor beta (TGF-β) and assume various elongated spindle shapes 6. Furthermore, the spindle-shaped cells 6 expressed α-SMA (alpha-actin-2, alpha smooth muscle actin) to a considerable extent, which leads to the development of posterior capsule opacification (PCO, cataracta secundaria). It can be seen in FIG. 3 that the spindle-shaped cells 6, which lead to fibrosis and wrinkling, also infiltrate the optical part 3 of the implant 1. What sometimes form are so-called Wedl cells 7, which form inflated, irregularly shaped fibers, tear and spread cell debris. These aberrant Wedl cells 7 and fragments thereof also collect in a disordered manner on the optical part 3 and form sites of clouding. Histopathologically, the Wedl cells 7 correspond to the clinically visible Hirschberg-Elschnig pearls, which are also responsible for the formation of the so-called Soemmering ring, which was first described in connection with eye trauma.

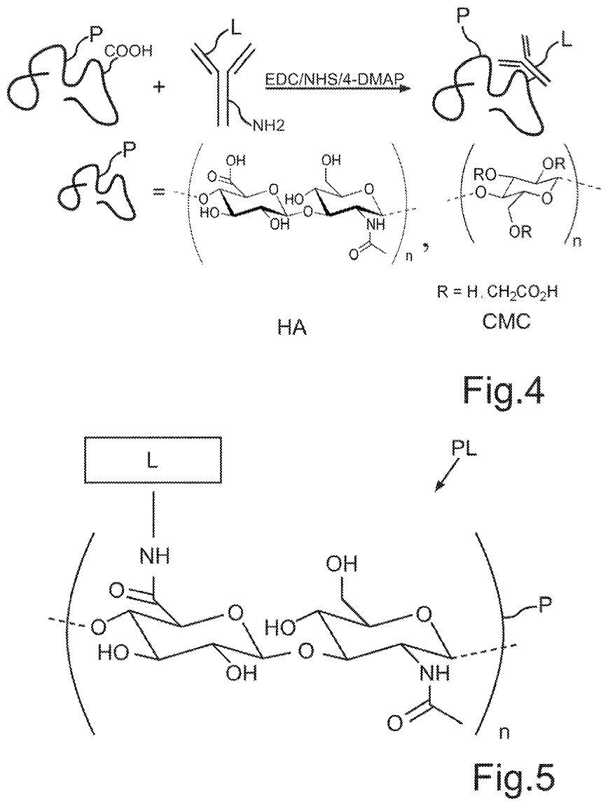

FIG. 4 shows a depiction of the principle of a reaction for covalently coupling a ligand L to a polymer or biopolymer, which, in the present case and by way of example, is a polysaccharide P, namely hyaluronic acid HA or carboxymethylcellulose CMC. The ligand L is, in some embodiments, also coupled with other polymers instead of the polysaccharide P. Hyaluronic acid belongs to the group of glycosaminoglycans, which are polysaccharides consisting of repetitive, linearly constructed and acidic disaccharide units. The disaccharide units of glycosaminoglycans generally include esters of a uronic acid. It is glucuronic acid in most cases, less frequently iduronic acid. The disaccharide units are linked to an amino sugar (for example, N-acetylglucosamine) via a 1,3-glycosidic bond. The formation of a chain of disaccharide units is achieved via 1,4-glycosidic bonds. Owing to relevant side groups (hydroxyl, carboxy or sulfate groups), the glycosaminoglycans are negatively charged. Glycosaminoglycans are divisible into sulfated and nonsulfated glycosaminoglycans. Hyaluronic acid (hyaluronan) is the only nonsulfated glycosaminoglycan and has free carboxy groups which, as a result of activation with the aid of N-hydroxysulfosuccinimide (NHS/sulfo-NHS) and subsequent carbodiimide-mediated (for example, 1-ethyl-3-(3-dimethylaminopropyl)carbodiimide hydrochloride/EDC and/or N′,N′-dicyclohexylcarbodiimide/DCC) reaction, can be covalently coupled with the ligand L. If necessary, 4-(dimethylamino)pyridine (4-DMAP) can be used as a nucleophilic catalyst in order to couple sterically demanding ligands L under mild conditions. Covalent coupling is in some embodiments analogously carried out with acid groups of carboxymethylcellulose.

In the embodiment shown in FIG. 4, the ligand L used is, by way of example, an anti-TGF-β antibody which is coupled to the polysaccharide P via a free amino group. Alternatively, in other embodiments, the ligand L is, for example, an anti-TGF-α antibody, an anti-interleukin-1 antibody, a Fab fragment, a single-chain variable fragment (scFv), or a multivalent antibody fragment (scFv multimer), and any combinations thereof, and it can be coupled with the polysaccharide P.

The resulting polysaccharide P coupled with the ligand L is shown schematically in FIG. 5, with an exemplary depiction of hyaluronic acid as polysaccharide P and an antibody, for example anti-TGF-β and/or anti-IL-1α, as ligand L. The polysaccharide-ligand compound PL is used in some embodiment for removal and/or biological deactivation of fibrinogens and/or cytokines with which an ophthalmological implant 10 comes or can come into contact when it has been implanted. The polysaccharide-ligand compound PL is in some embodiments present as a physiologically acceptable salt, in particular as a lithium salt. To this end, lithium hyaluronate, in an embodiment, is used as reactant and coupled with the ligand L. Alternatively, the polysaccharide-ligand compound PL is, in another embodiment, after production, converted into a physiologically acceptable salt, for example a lithium salt.

Without wishing to be bound by theory, it is assumed that topically administered alkali metal ions from the group consisting of Li⁺, K⁺, Rb⁺, and Cs⁺, can considerably slow down or even completely prevent the development of PCO and fibrosis, efficacy generally being the highest for Li⁺. The ions prevent the conversion of equatorial epithelial cells which were not removed during an eye operation into fibroblasts, fibroblasts being the main cause of PCO and fibrosis. The effect is presumably based on the ability of alkali metal ions of the group mentioned to stabilize the quiescent, polarized phenotype of lens epithelial cells. The epithelial cells in question therefore maintain the apical-basolateral polarity and cobblestone-like arrangement that are characteristic of lens epithelial cells. When applied locally, the alkali metal ions of the group mentioned also block the potent epithelial mesenchymal transition (EMT)-promoting effect of transforming growth factor beta (TGF-β) on lens epithelial cells. Experiments have shown that cells in TGF-β-treated explants progressively dissociated away from one another and assumed various elongated spindle shapes. Furthermore, the cells expressed α-SMA (apha-actin-2, alpha smooth muscle actin) to a considerable extent. These features are characteristic of the development of PCO. As a result of treatment with alkali metal ions of the group mentioned, it was possible to block the formation of α-SMA effectively and to stabilize the cells in the polarized, adherent, cobblestone-like monolayer. The counterions (anion) which can be used in relation to the alkali metal ion(s) (cation) used are in principle all suitable or pharmaceutically acceptable anions formed from atoms and/or molecules. Particular preference is given to chloride ions (Cl⁻). The active-substance delivery system can in general exclusively consist of a compound containing alkali metal ions of the group mentioned. Alternatively, further components can be provided, for example components for adjusting delivery of active-substance alkali metal ions or components in the form of binding or release agents.

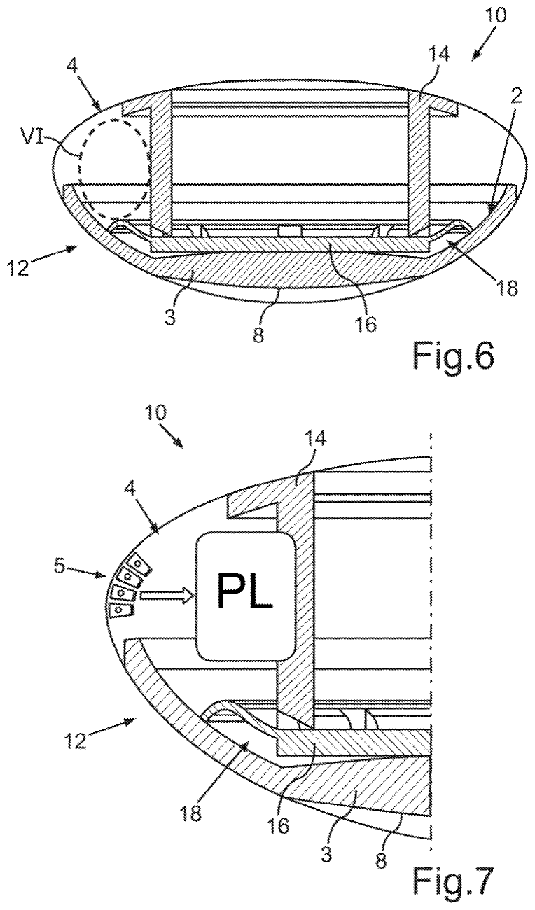

FIG. 6 shows a schematic lateral sectional view of one embodiment of an ophthalmological implant 10 that has been implanted in a capsular bag 4 of a patient. In the present case, the implant 10 is configured as an accommodating intraocular lens (IOL) and comprises a main body 12 having a haptic part 2 and an optical part 3 which has a lens body 8. Furthermore, the implant 10 comprises a hollow, centrally arranged cylinder 14 which, when implanted, applies a force to a spring-mounted membrane 16 in an axial manner with respect to a central axis of the implant 10. The membrane 16 is part of a wall of a gas-filled cavity 18 and delimits the cavity 18 in the direction of the cylinder 14, whereas the optical part 3 delimits the side of the cavity 18 that is facing away from the cylinder 14.

It can be seen that the haptic part 2 and the edge of the cylinder 14 have situated therebetween, in the axial direction, an encircling accommodation region, marked with a circle VI, into which equatorial lens epithelial cells (E cells) 5 can penetrate and can, as a result of secretion of cytokines and/or fibrinogens, for example of TGF-β, interleukin-1 and the like, cause cell proliferation, cell differentiation and infiltration of the implant 10 with fibroblasts and the like, which can consequently lead to PCO and fibrosis.

In order to prevent the occurrence of PCO and fibrosis, the polysaccharide-ligand compound PL is immobilized on the ophthalmological implant 10. In relation to this, FIG. 7 shows a schematic view of part of the ophthalmological implant 10, with immobilization of the polysaccharide-ligand compound PL in the accommodation region VI in order to bind and/or biologically deactivate cytokines and/or fibrinogens in the aqueous humor. The polysaccharide-ligand compound PL has been arranged in such a way that the accommodation ability of the implant 10 is not hindered. The polysaccharide-ligand compound PL has preferably been arranged in an encircling manner on the outer circumference of the cylinder 14 and thus in the vicinity of the E cells 5 in order to ensure particularly reliable and long-lasting protection. Alternatively, the polysaccharide-ligand compound PL has been arranged at one or more discrete sites on the outside of the implant 10. It is likewise conceivable that the implant 10 has been partially or completely coated with the polysaccharide-ligand compound PL.

In some embodiments, a layer system (not shown) having two or more layers is also provided. Irrespective of this, it is also possible to form the implant 10 from the polysaccharide-ligand compound PL at least in part. In addition to the polysaccharide-ligand compound PL, one or more further compounds are optionally provided, for example in order to achieve additional medical effects, to adjust chemical and/or mechanical properties and/or to bring about or assist immobilization on the implant 10.

For example, the polysaccharide P is in some embodiments bonded to the material of the implant 10 via 1,4-butanediol diglycidyl ether as a crosslinker. The crosslinking rate can be used to control or adjust the amount of ligands and thus to control the theoretically bindable and/or deactivatable amount of fibrinogens/cytokines and to control the biodegradability of the polysaccharide P (for example, by hyaluronidase)

In the nonimplanted state, the polysaccharide-ligand compound PL is in some embodiments nonhydrated or anhydrous, in particular lyophilized, in order to be able to compress the implant 10 as much as possible and to implant it through an appropriately small incision. Both polysaccharides P or other polymers and ligands L such as, for instance, antibodies are obtainable in anhydrous form, for example by lyophilization, and also storable for a long time as a covalent conjugate in this form. Hydration with a corresponding increase in volume then takes place automatically after implantation as a result of contact with aqueous humor. The covalent bonding of the ligand L and the immobilization of the polysaccharide P on the implant 10 prevent the aqueous humor from washing out the ligand L or the polysaccharide-ligand compound PL.

Alternatively or additionally, in an embodiment, the polysaccharide and/or another type of polymer, optionally in the form of a hydrogel or a foam, forms a matrix—in particular biodegradable matrix—in which the ligand L or multiple different ligands L are embedded and thereby immobilized in the matrix. The other type of polymer is, in some embodiments, a homopolymer or copolymer that has been optionally crosslinked. For example, the other type of polymer is an alginate. Alternatively or additionally, the matrix contains or consists of an alkali metal salt, for example lithium hyaluronate. As a result, what can be realized in addition to the immobilized ligand L is a kind of active-substance delivery system which can bind and/or deactivate cytokines and/or fibrinogens from the aqueous humor over an adjustable period of time. Furthermore, the ligand L immobilized in the matrix can also be released over time as a result of degradation of the matrix.

The parameter values specified in the documents to define process and measurement conditions for the characterization of specific properties of the subject matter of the described embodiments should also be considered to be encompassed herein in the context of deviations—for example due to measurement errors, system errors, weighing errors, DIN tolerances, and the like.

It is understood that the foregoing description is that of various embodiments and that various changes and modifications may be made thereto without departing from the spirit and scope as defined in the appended claims.

### LIST OF REFERENCE SIGNS

1 Implant (prior art)
2 Haptic part
3 Optical part
4 Capsular bag
5 E cells
6 Cells
7 Wedl cells
10 Implant according to the invention
12 Main body
14 Cylinder
16 Membrane
18 Cavity
VI Accommodation region
P Polysaccharide
L Ligand
PL Polysaccharide-ligand compound
HA Hyaluronic acid
CMC Carboxymethylcellulose

What is claimed is:

1. An ophthalmological implant, which comprises:
a main body, and
at least one ligand immobilized on the main body,
wherein the at least one ligand binds and/or deactivates at least one fibrinogen and/or cytokine when contacted with the at least one ligand and when the ophthalmological implant is implanted.

2. The ophthalmological implant of claim 1, wherein the ligand is embedded in a matrix and/or the ligand is covalently bonded to at least one polymer.

3. The ophthalmological implant of claim 2, wherein the at least one ligand is covalently coupled to the at least one polymer via a spacer.

4. The ophthalmological implant of claim 2, wherein the at least one polymer is a polysaccharide selected from of cellulose, a cellulose ether, a glycosaminoglycan, chondroitin sulfate, dermatan sulfate, heparin, heparan sulfate, keratan sulfate, alginic acid, polymannuronic acid, polyguluronic acid, polyglucuronic acid, amylose, amylopectin, callose, chitosan, polygalactomannan, dextran, xanthan gum, and/or a mixture thereof, and/or a physiologically acceptable salt thereof.

5. The ophthalmological implant of claim 1, wherein the main body further comprises at least one haptic part and at least one optical part.

6. The ophthalmological implant of claim 1, wherein the main body further comprises an accommodation region, and wherein the immobilized ligand is attached to the accommodation region.

7. The ophthalmological implant of claim 1, wherein at least part of a surface of the main body is coated with a layer system which comprises the immobilized ligand.

8. The ophthalmological implant of claim 7, wherein the layer system comprises at least two layers, wherein at least one of the at least two layers comprises the immobilized ligand and at least one of the at least two layers comprises a polymer selected from polyethylenimines, polyamines, and polyallylamines.

9. The ophthalmological implant of claim 1, wherein the at least one ligand further binds and/or deactivates TGF-β, TNF-α, and/or interleukin-1.

10. The ophthalmological implant of claim 1, wherein the at least one ligand is selected from antibodies, Fab fragments, single-chain variable fragments (scFv), and multivalent antibody fragments.

11. A method for producing an ophthalmological implant, which comprises: immobilizing at least one ligand on a main body, wherein at least one fibrinogen and/or cytokine is bound and/or deactivated on contacting the at least one ligand when the ophthalmological implant is implanted.

12. The method of claim 11, wherein the at least one ligand (L) is covalently coupled to a polysaccharide (P.

13. The method of claim 11, further comprising:
generating a layer system comprising at least two layers on a surface region of the main body, wherein at least one of the at least two layers comprises the immobilized ligand and at least one of the at least two layers comprises a polymer selected from polyethylenimines, polyamines, and polyallylamines.

14. The method of claim 11, wherein the main body further comprises an active-substance delivery system designed to deliver alkali metal ions selected from Li⁺, K⁺, Rb⁺, and/or Cs⁺, when the ophthalmological implant is implanted.

15. The ophthalmological implant of claim 2, wherein the at least one polymer is at least one biopolymer.

16. The ophthalmological implant of claim 4, wherein the at least one polymer is the cellulose ether substituted with methyl and/or ethyl and/or propyl groups.

17. The ophthalmological implant of claim 16, wherein the cellulose ether substituted with methyl and/or ethyl and/or propyl groups is hydroxypropylmethylcellulose, hydroxyethylmethylcellulose, and/or methylcellulose.

18. The ophthalmological implant of claim 4, wherein the polysaccharide is glycosaminoglycan, and wherein the glycosaminoglycan is hyaluronic acid.

19. The ophthalmological implant of claim 4, wherein the physiologically acceptable salt is an alkali metal salt.

20. The ophthalmological implant of claim 5, wherein the ligand is at least attached to the haptic part.

21. The ophthalmological implant of claim 6, wherein the ligand is covalently bonded to the main body.

22. The ophthalmological implant of claim 10, wherein the ligand is an antibody, and wherein the antibody is an anti-TGF-β antibody, anti-TGFα antibody, and/or anti-interleukin-1 antibody.

23. The method of claim 12, wherein the covalent coupling is performed by:
providing the ligand, wherein the ligand comprises at least one amino group;
providing the polysaccharide, wherein the polysaccharide comprises at least one carboxylic acid group;
activating, at least partially, the carboxylic acid group of the polysaccharide; and
coupling the activated carboxylic acid group with the at least one amino group of the ligand.

24. A method of treating and/or reducing the occurrence of posterior capsule opacification or cataracta secundaria, which comprises:
implanting the ophthalmological implant of claim 1 into an eye of a subject in need thereof.

25. The method of claim 24, wherein the subject has had one or more cataract operations in the eye.

26. The method of claim 24, wherein the subject is human.

27. The method of claim 24, wherein the ligand is embedded in a matrix and/or the ligand is covalently bonded to at least one polymer.

28. The method of claim 27, wherein the at least one ligand is covalently coupled to the at least one polymer via a spacer.

29. The method of claim 27, wherein the at least one polymer is a polysaccharide selected from of cellulose, a cellulose ether comprising methyl and/or ethyl and/or propyl groups, a glycosaminoglycan, chondroitin sulfate, dermatan sulfate, heparin, heparan sulfate, keratan sulfate, alginic acid, polymannuronic acid, polyguluronic acid, polyglucuronic acid, amylose, amylopectin, callose, chitosan, polygalactomannan, dextran, xanthan gum, and/or a mixture thereof, and/or a physiologically acceptable salt thereof.
